(19) United States
(12) Patent Application Publication
(10) Pub. No.: US 2021/0220498 A1
(43) Pub. Date: Jul. 22, 2021

(54) DISINFECTING LOCKER DEVICES AND SYSTEMS

(71) Applicant: SMIOTA, INC., Pleasanton, CA (US)

(72) Inventor: MANJUNATHA KASHI, MILPITAS, CA (US)

(21) Appl. No.: 17/221,834

(22) Filed: Apr. 4, 2021

Related U.S. Application Data
(63) Continuation-in-part of application No. 16/209,191, filed on Dec. 4, 2018.
(60) Provisional application No. 63/005,312, filed on Apr. 4, 2020.

(51) Int. Cl. A61L 2/10; A61L 2/24

(52) U.S. Cl. CPC A61L 2/10; A61L 2/24; A61L 2202/23; A61L 2202/122; A61L 2202/14; A61L 2202/11

(57) ABSTRACT

Disclosed herein are devices, systems and methods for disinfecting parcels stored within disinfecting smart locker devices.

### CROSS-REFERENCE TO RELATED APPLICATION

This application claims priority to and claims the benefit of priority to U.S. Provisional Patent Application No. 63/005,312 filed on Apr. 4, 2020, and entitled “DISINFECTING LOCKER DEVICES AND SYSTEMS”, which claims priority to U.S. patent application Ser. No. 16/209,191 filed on Dec. 4, 2018, and entitled “GENERATING EXCHANGE LOCATIONS”. The entirety of the disclosure of the aforementioned application is considered part of, and is incorporated by reference in, the disclosure of this application

### BACKGROUND

Currently, last mile delivery has received notable attention, specifically with respect to parcel deliveries. With the rise of e-commerce transactions, and consumer preference to receive products via parcel, there is a growing demand for package storage solutions including smart locker technologies. Accordingly, smart lockers are used more frequently to facilitate the delivery and receipt of parcels. Given, the increase in parcel transactions using smart locker technologies, there is also an increase in the transmission of germs such as bacteria and virus's present on the parcel surfaces. For instance, virus's such as SARS-CoV-2 and COVID-19 (also referred to as coronavirus) can survive on certain material surfaces such as skin, fabric, wood, plastic, steel and cardboard. As such, viruses and bacteria are transmittable to people while living on the surface of parcels. Given, the dangers posed by germ transmission on the surface of packages, there need to be technological solutions to the problems of germ transmission on the surface of packages.

### BRIEF DESCRIPTION OF THE DRAWINGS

FIG. 1 illustrates a flow diagram of an example, non-limiting method 100 that facilitates a disinfecting of parcels with a disinfecting smart locker device in accordance with one or more embodiments described herein.

### DETAILED DESCRIPTION

The following detailed description is merely illustrative and is not intended to limit embodiments and/or application or uses of embodiments. Furthermore, there is no intention to be bound by any expressed or implied information presented in the preceding Background or Summary sections, or in the Detailed Description section. One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

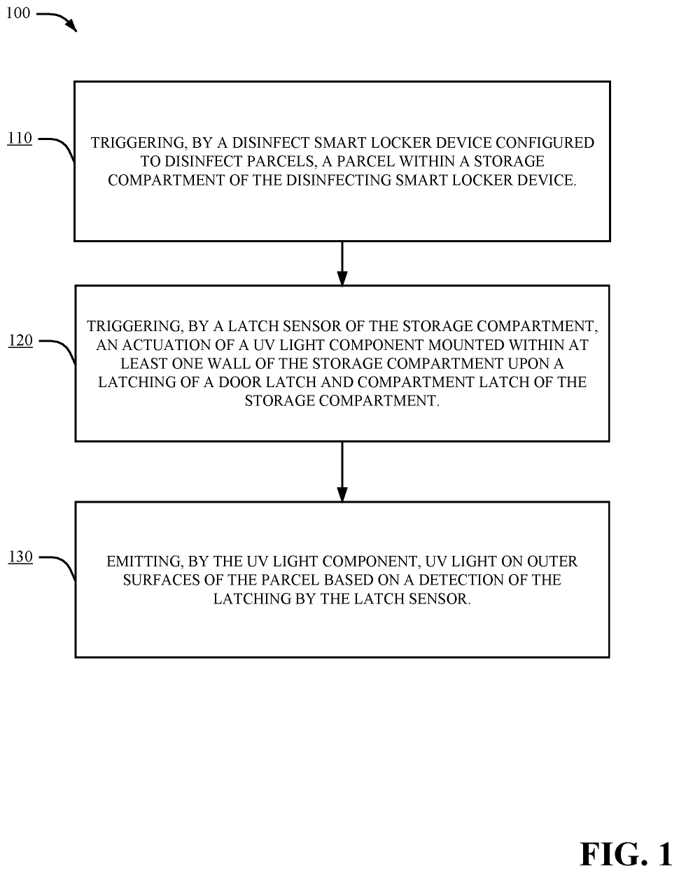

Turning now to FIG. 1, illustrated is a non-limiting method 100 that facilitates an analysis of one or more package in accordance with one or more embodiments described herein. In an aspect, the method includes reference numeral 110 that includes receiving, by a disinfecting smart locker device configured to disinfect parcels, a parcel within a storage compartment of the disinfecting smart locker device. At reference numeral 120, the method includes triggering, by a latch sensor of the storage compartment, an actuation of a UV light component mounted within at least one wall of the storage compartment upon a latching of a door latch and compartment latch of the storage compartment. At reference numeral 130, the method includes emitting, by the UV light component, UV light on outer surfaces of the parcel based on a detection of the latching by the latch sensor.

In an aspect, disclosed are disinfecting smart lockers configured to store parcels within compartments of the smart locker and disinfect the surfaces of the stored parcels. Furthermore, in an aspect, the disinfecting smart lockers can comprise a locker housing that is transparent based on a transparent material composition. For instance, the disinfecting smart locker device can have a housing comprised of plexiglass, borosilicate glass, polycarbonate, or optical silicone. In various embodiments, the transparent material can be selected based on thermal properties, optical properties, mechanical properties or other such considerations. For instance, plexi-glass can withstand pressure, is rigid, and won't deform in shape if under pressure.

In an aspect, all portions of the disinfecting smart locker housing and compartment can be transparent. For instance, this includes, all exterior panels of the locker housing, all front compartment door panels, all locker shelving, all locker compartment walls, and any other portion of the disinfecting smart locker such that the transparent material allows natural ultraviolet light emissions from the sun to impact the surfaces of the parcels stored in the compartments of the smart locker device. In other non-limiting embodiments, the disinfecting smart locker device can include housing and/or base plates (e.g., floor and roof of storage compartments) that parcels rest upon patterned as a grill, pin-hole pattern, sieve pattern, or other porous composition that allows UV light emissions to pass through such storage compartment enclosure wall (e.g., side walls, roof, floor, etc.)

Furthermore, in other embodiments, the smart locker compartments can comprise ultraviolet light lamp fixtures mounted within the locker compartments to further disinfect package surfaces. For instance, an ultraviolet light fixture can be mounted to all walls of the locker compartments. Furthermore, the ceiling of a lower locker can be mounted with more than one ultraviolet (UV) light fixture with at least one facing upward and another facing downward such that ultraviolet light can pass through the luminescent ceiling of the compartment above.

In another aspect, the ultraviolet lights lamps within the smart locker compartments can be triggered to emit ultraviolet light (e.g., for predetermined time durations based on contamination levels and other such factors) upon the actuation of a switch or other trigger (e.g., mechanical or electrical) based on the detection that a latching mechanism between a compartment door and the locker panel interfacing with the compartment door are fastened to one another based on a latching mechanism (e.g., cam latches, compression latches, slam latches, draw latches, etc.). Upon the detection that a latching has occurred, the UV light fixture can emit ultraviolet light onto the surface of the parcel to disinfect such surfaces by killing bacteria, viruses, and other contaminants residing on the parcel. Furthermore, the mounted UV light fixtures can emit UV light bi-directionally.

The triggering of UV emissions during a storage locker closing event provides a safety mechanism for users such that UV exposure risks are mitigated due to the UV lamps operated only when the compartments are closed (e.g., UV light emissions contained within the storage compartment). In some embodiments, the disinfecting smart locker devices can comprise housings that are not transparent such the UV light emissions from mounted lamps are further contained within the storage walls (e.g., UV light emissions don't emit outside of the inner walls of the storage compartment). In an aspect, the UV lamps can emit UV light emitting at various wavelengths. In a non-limiting embodiment, the UV lamps can emit far ultraviolet C (far UV-C) light in continuous low doses that emits light between the 315 nm and 400 nm range. In an aspect, the far UV-C light is configured to emit within a storage compartment of the locker. In an aspect, the system can employ the UV light emissions upon a closing of the storage compartment door. As such, the closed storage compartment of the disinfecting smart locker device can safely disinfect packaged within contained within the storage compartment and prevent users from exposure to the UV light emissions.

In another instance, a UV lamp mounted to the roof of a compartment can face upward to emit UV light on the bottom of a parcel sitting in a compartment above the UV lamp and given that the above compartment has a floor comprised of a transparent material, the UV light can penetrate through the transparent material and impact the bottom surface of the parcel. Furthermore, in an aspect, another UV lamp mounted to the roof of the same compartment can face downward to emit UV light on the top surface of a parcel sitting within that same compartment. Accordingly, any compartment well of the disinfecting smart locker device can include UV lamp lights mounted in a manner to direct UV light in opposite directions such that all parcels within the smart locker device can be disinfected.

In other embodiments, disinfecting smart locker devices can employ aerosol spraying mechanisms within storage compartments to further disinfect surfaces of parcels. In an aspect, the system can employ the aerosol spray mechanism upon a closing of the storage compartment door. As such, the closed storage compartment of the disinfecting smart locker device can safely disinfect packaged within contained within the storage compartment and prevent users from exposure to the aerosolized disinfecting agents. In another aspect, the systems (e.g., server devices) communicatively coupled to the disinfecting smart locker devices can detect (e.g., via sensors, machine vision implementations, equipment maintenance integrations, etc.) when disinfecting equipment employed by the disinfecting smart locker device requires replacement, maintenance and/or repair.

For instance, in the event an aerosolized disinfecting component of the disinfecting smart locker device is low on disinfecting agent or the aerosolized release mechanism is not working properly, the smart locker device (e.g., sensors) can detect such issue and trigger notifications to the server device in order to initiate repair, maintenance, or other remedies to be undertaken. Furthermore, the server device can employ system mechanisms to require maintenance, repair, or replacement operations to be undertaken. For instance, if new UV lamps are installed within locker compartments on X date and they have a lifespan of 3 years, the server device may initiate a replacement requirement after 30 months has elapsed from X date. Other such system implementations can facilitate proper operability of the disinfecting smart locker device.

In another aspect, the disinfecting smart locker device can be configured to detect (e.g., using sensors) various agents present on the surface of parcels prior to decontamination and after decontamination. For instance, scanning mechanisms can be employed to detect viral agents on the surface of parcels. Furthermore, a platform system executing on one or more server communicative coupled to the smart locker device can execute operations of the disinfecting smart locker devices such as employing UV light sensors, generating data associated with the use of such components and the smart locker device, authenticating disinfecting activities performed by the device, and interacting with other devices (e.g., user mobile devices, enterprise devices, hospital devices, dispensaries, urgent care devices, etc.).

In another aspect, the disinfecting smart locker devices can utilize inventory management systems to ensure that integrate with the smart locker devices to propagate supplies in based on the needs of smart locker users. Furthermore, in emergent situations such as pandemics in order to satisfy the needs of critical products to users and track the deployment of such products (e.g., within parcels) within storage compartments in accordance with the identity of a user device accessing such products, frequency of access of such products, and determinations of priority based on rationing algorithms.

As such, the disinfecting locker devices can utilize systems that determine when inventory is low, how to ration such inventory, when to replenish such inventory, ideas as to how to replenish such inventory quickly, when to start and stop production, stocking decisions based on sales costs, deploying parcels in accordance with rationing policies and re-order policies. As such, not only can disinfecting locker devices reduce contaminants on the surface of parcels delivered, stored, and accessed with the smart locker devices, but also inform enterprise systems (e.g., hospital systems, retail store enterprise systems, urgent care facility systems, etc.) as to the identity of users accessing critical goods and operating in accordance with rules that allow for the most users to access critical items in a time of need.

For instance, a disinfecting locker device can contain parcels that store essential supplies for users during pandemics or emergent situations such as protective equipment (e.g., masks, respirators, etc.), sanitizers (e.g., hand sanitizers, disinfectant sprays, etc.), home supplies (e.g., toilet paper), food rations (e.g., meat, non-perishable items, bread, etc.), medical kits, and other such items. Furthermore, the platform systems communicatively coupled to the disinfecting smart locker devices can monitor and track the identity of the users whom access such essential goods and the frequency with which such users access such goods. The system can also limit the ability for users to access such parcels prior to a threshold number of unique identity users accessing such parcels. Accordingly, the systems and devices herein can create a utility model that allows the most users to obtain essential items as frequently as possible. Furthermore, the systems can be utilized by enterprises or government users to ensure that products within parcels are reaching the majority of users within an emergency-stricken population. Accordingly, such solutions can also reduce queue's associated with panic purchasing such that disinfecting smart locker devices can appropriately schedule the pick-up of parcels by users in an ordered and staggered manner and efficiently governs the user pick-up process.

In another aspect, the disinfecting smart locker devices can utilize technologies that allow users to utilize wireless personal area network technologies configured to transmit radio signals for detection by user devices (e.g., mobile devices). As such, the disinfecting smart locker device can be configured to include a wireless personal area network token (e.g., Bluetooth Low Energy (BLE) token) (referred to as a BLE token) that is equipped with radio broadcasting technologies. Furthermore, a user smart phone device can execute an application that emits a BLE signal, such that the BLE token coupled to the disinfecting smart locker device can detect. Accordingly, the application executing on the user smart phone device can transmit a request to a server device for packages corresponding to the respective user and stored within the storage compartment of the smart locker device.

Furthermore, the detection of the smart phone device can trigger a disinfecting operation of the smart locker device. For instance, the smart locker device can trigger an emission of UV-C light within the storage compartment containing the parcel associated with a smart phone device application detected by the BLE token of the disinfecting smart locker device. In an aspect, the server, upon receiving a request for packages from the smart phone device, can transmit an operable capability to the user smart phone device that allows the smart phone device to execute an unlock operation of the storage compartment containing the parcel associated with the smart phone device.

The smart phone device can initiate an unlock instruction (e.g., input received at the smart phone user interface initiating the unlocking) by transmitting the unlock request to the server device. Furthermore, the server device can transmit the signal and an authenticated instruction to the disinfecting locker device to unlock the storage compartment containing the parcel. Furthermore, the disinfecting smart locker device opens the appropriate storage compartment and transmits an update to the server device. The server device transmits updates to the application executing on the smart phone device. Furthermore, the user can retrieve the disinfected parcel and close the door to the storage compartment. The disinfecting smart locker device transmits updates to the server device that the door to the compartment has been closed. The server device updates the application executing on the smart phone device and removes unlock permissions (e.g., of the storage compartment) associated with the application.

By employing the BLE technology implementation, the disinfecting smart locker device, allows users to interact with storage compartments without ever having to touch the compartment or in a manner that minimizes physical contact (e.g., touching of the storage compartment). For instance, a user only need interact with the application executing on its mobile device to successfully gain access to a disinfected parcel stored within the storage compartment. In an aspect, such limited contact requirement can minimize the transmission of viruses (e.g., severe acute respiratory syndrome coronavirus 2 (SARS-CoV-2), COVID-19, etc.), bacteria, or other agents transmittable via touching. As such, the BLE token technology implementation can mitigate transmission of viruses, agents, or bacteria from the smart locker device to a user via minimizing the need for physical touching of the disinfecting smart locker device. This technology implementation also eliminates the need for a user to interface with a smart locker device console which often could be used by any number of users capable of transmitting viruses or bacteria to such console.

In other embodiments, the disinfecting smart locker devices can integrate with hospital systems to enable the disinfecting of medical professional user items. For instance, physicians and other medical professionals can store personal items (e.g., shoes, coats, gloves, gowns, personal protective equipment (PPE) within storage compartments of the disinfecting smart locker devices and the device can employ UV lamps (e.g., emitting UV-C light) emitting UV light to disinfect such items at various times throughout a work day. As such, medical professionals can utilize the disinfecting smart locker devices to reduce the prospect of transmitting viral and bacterial agents to themselves and others via PPE. As such, the disinfecting smart locker devices can assist with protecting higher risk users (e.g., medical professionals) especially in environments where shortages of medical supplies (e.g., PPE) exist to necessitate re-use of potentially contaminated medical supplies.

In an aspect, the server device communicatively coupled to each disinfecting smart locker device can generate and monitor key performance metrics related to use of disinfecting smart locker device. For instance, the server device can track the number of times an item (e.g., medical supply, parcel, etc.) has undergone a disinfecting UV light treatment. Accordingly, a user (e.g., medical professional) can comprehend the need to dispose an item (e.g., medical supply) after a maximum number of safe uses has occurred. Furthermore, disinfecting smart locker device can generate such key performance metrics employing machine learning techniques, data querying techniques, and entity relationship generation to identify such performance metrics over a period of time and a period of users of the disinfecting smart locker devices.

Turning now to FIG. 1, illustrated is a method 100 of disinfecting parcels by a disinfecting smart locker device. At reference numeral 110, a disinfecting operation is triggered, by a disinfecting smart locker device configured to disinfect a parcel within a storage compartment of the disinfecting smart locker device. At reference numeral 120, a latch sensor of the storage compartment triggers an actuation of a UV light component mounted within at least one wall of the storage compartment upon a latching of a door latch and compartment latch of the storage compartment. At reference numeral 330, the UV light component emits UV light on outer surfaces of the parcel based on a detection of the latching by the latch sensors

1. A method of disinfecting a package comprising:
receiving, by a disinfecting smart locker device configured to disinfect parcels, a parcel within a storage compartment of the disinfecting smart locker device;
triggering, by a latch sensor of the storage compartment, an actuation of a UV light component mounted within at least one wall of the storage compartment upon a latching of a door latch and compartment latch of the storage compartment; and
emitting, by the UV light component, UV light on outer surfaces of the parcel based on a detection of the latching by the latch sensor.

2. The method of claim 1, further comprising directing, by a transparent housing of the storage compartment, naturally generated ultraviolet light toward the parcel surfaces within the storage compartment through the transparent housing.
